(19) United States
(12) Patent Application Publication
(10) Pub. No.: US 2021/0082543 A1
(43) Pub. Date: Mar. 18, 2021

(54) Method for establishing chart for designing mechanical properties of cement stones in large-scale fracturing oil well

(71) Applicant: Southwest Petroleum University, Chengdu (CN)

(72) Inventors: Hu Yin, Chengdu (CN); Qian Li, Chengdu (CN); Xiuwen Zhao, Chengdu (CN); Wenfeng Yin, Chengdu (CN)

(73) Assignee: Southwest Petroleum University

(21) Appl. No.: 17/105,481

(22) Filed: Nov. 25, 2020

(30) Foreign Application Priority Data
Sep. 7, 2020 (CN) 202010927277.4

(51) Int. Cl. G16C 60/00; E21B 33/02; G16C 20/30; G16C 20/10

(52) U.S. Cl. CPC G16C 60/00; E21B 33/02; E21B 2200/20; G16C 20/10; G16C 20/30

(57) ABSTRACT

A method for establishing a chart for designing mechanical properties of cement stones in a large-scale fracturing oil well is provided, including steps of: establishing a stress distribution model of a cement sheath based on a theory of elasticity and thick-walled cylinder; establishing a cement sheath integrity prediction model based on the cement sheath failure criterion and the stress increment distribution state of the cement sheath; establishing a cement sheath integrity control method based on the cement sheath stress analysis model and the cement sheath integrity prediction model; establishing a functional relationship between cement stone mechanical parameters and strength parameters based on the cement sheath integrity control method; and establishing a cement stone performance index control chart based on the functional relationship between the cement stone mechanical parameters and strength parameters.

### CROSS REFERENCE OF RELATED APPLICATION

The present application claims priority under 35 U.S.C. 119(a-d) to CN 202010927277.4, filed Sep. 7, 2020.

### BACKGROUND OF THE PRESENT INVENTION

### Field of Invention

The present invention relates to a method for establishing a chart for designing mechanical properties of cement stones in large-scale fracturing oil well, which belongs to the technical field of oil and gas drilling.

### Description of Related Arts

In the process of large-scale fracturing of horizontal wells, under the action of high internal pressure, the cement sheath may undergo tensile failure, leading to failure of inter-section isolation and affecting the effect of staged fracturing. The cement sheath may also undergo plastic deformation and cannot be recovered after fracturing, causing the cement sheath casing interface to peel off to form micro-annular gaps. The fracturing fluid may flow through the fractured part of the cement sheath to the fractured section, reducing the effect of low pressure cracking. Therefore, in order to achieve the desired effect of large-scale fracturing of horizontal wells, the cement pastes need to have better mechanical properties. Cement stone must not only have a certain compressive strength, but also have deformation ability. The relevant standards for oil and gas well cement slurry design only stipulate that the compressive strength of horizontal well cement paste shall not be less than 14 MPa in 24 hours, which may be not capable of meeting the sealing requirements of large-scale horizontal well fracturing.

Therefore, many scholars have carried out research and exploration on the integrity of the cement sheath. The research results show that in order to ensure the integrity of the cement sheath, cement stones should have the characteristics of high strength and low modulus. However, the performance indexes of the cement stones are not clearly proposed and it is difficult to be actually applied on site. Therefore, there is an urgent need for a design method of cement stone performance indicators that is capable of being applied to the actual field.

### SUMMARY OF THE PRESENT INVENTION

The invention mainly overcomes the shortcomings in the prior art, and provides a method for establishing a chart for designing mechanical properties of cement stones in a large-scale fracturing oil well.

In order to solve the technical problems mentioned above, technical solutions provided by the present invention are as follows. A method for establishing a chart for designing mechanical properties of cement stones in a large-scale fracturing oil well, comprising steps of:

S1: establishing a stress distribution model of a cement sheath based on a theory of elasticity and thick-walled cylinder;

S2: establishing a cement sheath integrity prediction model based on the cement sheath failure criterion and the stress increment distribution state of the cement sheath;

S3: establishing a cement sheath integrity control method based on the cement sheath stress distribution model and the cement sheath integrity prediction model;

S4: establishing a functional relationship between cement stone mechanical parameters and strength parameters based on the cement sheath integrity control method; and

S5: establishing a cement stone performance index control chart based on the functional relationship between the cement stone mechanical parameters and strength parameters.

Preferably, the a specific establishing process of the step S1 comprises steps of:

S11: by Ariy's stress function, deriving the stress increment distribution function expression of the casing-cement sheath-formation system during the large-scale fracturing process based on the elasticity thick-walled cylinder theory;

S12: calculating the displacement increment function expression of the casing-cement sheath-stratum surrounding rock combination during the large-scale fracturing process according to the elasticity thick-walled cylinder theory; and

S13: calculating the unknown parameters in the stress increment distribution function expression and the displacement increment function expression using the conditions of equal stress increments at the boundary of the casing, cement sheath, and formation and continuous displacement, and then bringing them into the stress increment distribution function expression and displacement increment function expression of the casing-cement sheath-formation surrounding rock combination, and the stress increment distribution and displacement increase of the casing-cement sheath-formation surrounding rock combination is obtained.

Preferably, the stress increment distribution function expression comprises a circumferential stress increment expression, a radial stress increment expression, and a shear stress increment expression.

Preferably, the cement sheath failure criterion comprises a cement sheath tensile failure criterion and a cement sheath interface peeling failure criterion.

Preferably, the cement sheath integrity prediction model comprises a cement sheath tensile failure prediction model and a cement sheath interface peeling failure prediction model;

the tensile failure prediction model of the cement sheath is based on the tensile failure criterion of the cement sheath; if the circumferential stress increment of the inner wall of the cement sheath is smaller than the tensile strength of the cement stone, the tensile failure will not occur; and

the cement-to-interface peeling failure prediction model is based on the cement sheath interface peeling failure criterion, if the radial displacement increment of the inner wall of the cement stone is less than the yield strength of the cement stone, the interface peeling failure of the cement stone will not occur.

Preferably, the cement sheath integrity control method comprises following steps of:

A. obtaining current well casing, formation mechanical parameters, size parameters, and wellhead pressure increment;

B. setting the Young's modulus and Poisson's ratio of the mechanical parameters of the cement sheath, simulate the stress state of the cement sheath according to the stress distribution model of the cement sheath, and calculate the circumferential stress increment and radial stress increment of the cement sheath; and

C. based on the cement sheath integrity prediction model, under the current value of cement stone Young's modulus and Poisson's ratio, in order to avoid damage to the cement sheath, setting the cement stone tensile strength greater than the circumferential stress increment of the inner wall of the cement sheath, and the cement stone yields, and the strength greater than the increase in radial stress on the inner wall of the cement sheath.

Preferably, the functional relationship between the mechanical parameters and strength parameters of the cement stone includes the binary function relationship between the cement stone Young's modulus, Poisson's ratio, and the cement stone's tensile strength, and the cement stone Young's modulus, Poisson's ratio, Binary function relationship between the yield strength of cement stone.

Preferably, the cement stone performance index control plate comprises a plate to avoid tensile failure of cement stone and a plate to avoid peeling failure of cement stone.

Cementing cement is mainly used to seal the annulus between the casing and the wellbore. If the mechanical properties of the cement are not designed properly, the cement sheath may be damaged or the cementation interface may be peeled off during the fracturing process, resulting in fracturing channeling and fracturing effect is damaged. Under certain working conditions, the three mechanical parameters of cement stone's elastic modulus, Poisson's ratio and strength jointly affect the integrity of the cement sheath, and the reasonable value ranges of these three parameters affect each other.

Use this chart to quickly determine the mechanical parameters of cement stone that meet the requirements. At the same time, this chart can be extended and applied to other types of oil and gas well cement stone mechanical properties design. For example, the reasonable design of cement stone mechanical properties used in gas storage wells can effectively prevent plastic deformation of the cement sheath body during gas injection and pressurization in the gas storage wellbore, and avoid micro-annular gaps during unloading to cause gas leakage.

The present invention has the following beneficial effects: The present invention establishes a cement stone performance index control chart containing three parameters of cement stone Young's modulus, Poisson's ratio and cement stone strength. The performance range of cement stone determined by the control chart is greatly improved. The integrity of the cement sheath during large-scale fracturing has a certain theoretical guiding role.

### BRIEF DESCRIPTION OF THE DRAWINGS

FIG. 1 is a schematic flow chart of the present invention for establishing a cement stone performance index control chart.

FIG. 2 is a schematic diagram of the stress distribution model of the cement sheath of the present invention.

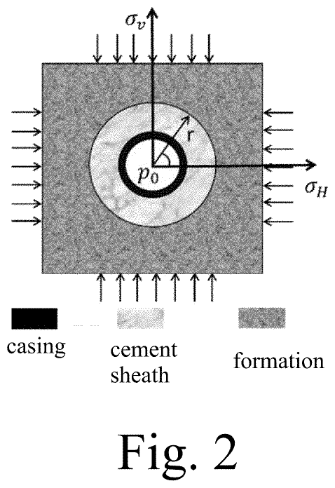

FIG. 3 is a cement stone performance index control chart established by the present invention to avoid tensile failure of cement stone.

FIG. 4 is a cement stone performance index control chart established by the present invention to avoid interfacial peeling damage of cement stone.

### DETAILED DESCRIPTION OF THE PREFERRED EMBODIMENT

The present invention will be further explained below in conjunction with the embodiments and the drawings.

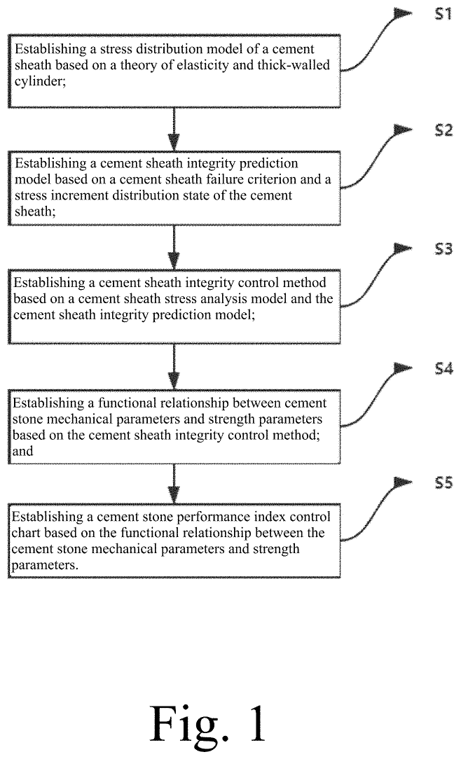

As shown in FIG. 1, the method for establishing a chart for designing mechanical properties of cement stones in large-scale fracturing oil well of the present invention comprises the following steps of:

S1. Establish a stress distribution model of cement sheath based on the theory of elasticity thick-walled cylinder.

The conditions for establishing the stress distribution model of the cement sheath are as follows.

(1) The casing and the surrounding rock of the formation are linear elastic materials.

(2) The cement sheath is an elasto-plastic material but the stress is not enough to cause plastic deformation of the cement sheath.

(3) Cementing quality is excellent.

(4) Casing-cement sheath-stratum and wellbore are concentric rings.

(5) There is no initial stress in the cement sheath;

(6) Only the influence of stress increment on cement sheath during fracturing is considered.

The specific establishment process comprises steps of:

S11. By Ariy's stress function to derive the stress increment distribution function expression of the casing-cement sheath-stratum surrounding rock combination during the large-scale fracturing process based on the elasticity thick-walled cylinder theory (Equation 3).

φ=(Ar⁴+Br²+C+Dr⁻²)cos 2ƒ+Fr² ln r+Hr²+K ln r+M (1)

The stress components represented by the stress function in polar coordinates are as follows:

$$\{ \begin{matrix}{σ}_{r} = \frac{1}{r} \frac{\partial ϕ}{\partial r} + \frac{1}{{r}^{2}} \frac{{\partial }^{2} ϕ}{\partial {θ}^{2}} \\ {σ}_{θ} = \frac{{\partial }^{2} ϕ}{\partial {r}^{2}} \\ {τ}_{r θ} = - \frac{\partial ϕ}{\partial r} ( \frac{1}{r} \frac{\partial ϕ}{\partial θ} )\end{matrix} \qquad ( 2 )$$

According to the single displacement condition, F=0 can be obtained; by integrating equation (1) into equation (2), the stress increment distribution function of the casing-cement sheath-formation surrounding rock combination can be obtained during the large-scale fracturing process Expression, its stress increment distribution function expression includes circumferential stress increment expression, radial stress increment expression, and shear stress increment expression;

$$\{ \begin{matrix}Δ {σ}_{ri} ( r ) = 2 {H}_{i} + {K}_{i} {r}^{- 2} - ( 2 {B}_{i} + \frac{4 {C}_{i}}{{r}^{2}} + \frac{6 {D}_{i}}{{r}^{4}} ) cos 2 θ \\ Δ {σ}_{θ i} ( r ) = 2 {H}_{i} - {K}_{i} {r}^{- 2} + ( 12 {A}_{i} {r}^{2} + 2 {B}_{i} + \frac{6 {D}_{i}}{{r}^{4}} ) cos 2 θ \\ Δ {τ}_{r θ i} ( r ) = ( 6 {A}_{i} {r}^{2} + 2 {B}_{i} - \frac{2 {C}_{i}}{{r}^{2}} - \frac{6 {D}_{1}}{{r}^{4}} ) sin 2 θ\end{matrix} \qquad ( 3 )$$

wherein: Aᵢ, Bᵢ, Cᵢ, Dᵢ, Hᵢ, Kᵢ are unknown parameters; r is the inner radius of the medium material, mm; Δσᵣ, (r), Δσ_{θi}(r), and Δτ_{rθi}(r), are radial stress increments circumferential stress increments, and shear stress increments respectively with a unit MPa; i=1, 2, 3 respectively represent casing and cement Surrounding rock of ring and stratum; θ is the well circumference angle with a unit °.

S12. Calculate the displacement increment function expression (Equation 5) of the casing-cement sheath-stratum surrounding rock combination body during the large-scale fracturing process according to the elasticity thick-walled cylinder theory (Equation 5), the displacement increment function expression includes Radial displacement incremental expression, circumferential displacement incremental expression.

The geometric equation of the plane strain problem in polar coordinates is as follows:

$$\{ \begin{matrix}{ɛ}_{r} = \frac{\partial {u}_{r}}{\partial r} = \frac{1 - {v}^{2}}{E} ( {σ}_{r} - \frac{v}{1 - v} {σ}_{θ} ) \\ {ɛ}_{θ} = \frac{{u}_{r}}{r} + \frac{1}{r} \frac{\partial {u}_{θ}}{\partial θ} = \frac{1 - {v}^{2}}{E} ( {σ}_{θ} - \frac{v}{1 - v} {σ}_{r} )\end{matrix} \qquad ( 4 )$$

Incorporating equation (3) into equation (4), the displacement increment caused by the increase in wellhead pressure is the displacement increment of the casing, cement sheath, and formation:

$$\{ \begin{matrix}Δ {u}_{ri} ( r ) = {G}_{i} [ 2 {H}_{i} ( 1 - 2 {v}_{i} ) r - {K}_{i} {r}^{- 1} ] - 2 {G}_{i} [ 2 {v}_{i} {A}_{i} {r}^{3} + {B}_{i} r - ( 1 - 2 {v}_{i} ) \frac{2 {C}_{i}}{r} - \frac{{D}_{i}}{{r}^{3}} ] cos 2 θ \\ Δ {u}_{θ i} ( r ) = 2 {G}_{i} [ ( 3 - 2 {v}_{i} ) {A}_{i} {r}^{3} + {B}_{i} r - ( 1 - 2 {v}_{i} ) \frac{2 {C}_{i}}{r} + \frac{{D}_{i}}{{r}^{3}} ] sin 2 θ\end{matrix} \qquad ( 5 )$$

wherein: Δuᵣᵢ(r) and Δu_{θi}(r) are the radial displacement increment and the circumferential displacement increment, respectively, μm; vᵢ is the Poisson's ratio of the dielectric material, dimensionless; Eᵢ is the Young's modulus of the dielectric material, GPa; Gᵢ=(1+vᵢ)/Eᵢ is the shear modulus of the dielectric material, GPa.

S13. Solve the unknown parameters in the stress increment distribution function expression and the displacement increment function expression using the conditions of equal stress increments at the boundary of the casing, cement sheath, and stratum and continuous displacement.

The boundary conditions are:

On the inner wall of the casing, the boundary conditions are:

$$\{ \begin{matrix}{Δσ}_{r 1} {\vert}_{r = {r}_{1}} = Δ {p}_{0} \\ {Δτ}_{r θ 1} {\vert}_{r = {r}_{1}} = 0\end{matrix} \qquad ( 6 )$$

At the outer boundary r=r₄ of the formation:

$$\{ \begin{matrix}Δ {σ}_{r} = - \frac{1}{2} ( {σ}_{v} - {σ}_{H} ) cos 2 θ \\ Δ {σ}_{θ} = \frac{1}{2} ( {σ}_{v} - {σ}_{H} ) cos 2 θ \\ {Δτ}_{r θ} = \frac{1}{2} ( {σ}_{v} - {σ}_{H} ) sin 2 θ\end{matrix} \qquad ( 7 )$$

wherein: Δp₀ is the wellhead pressure increase, MPa; σᵥ is the vertical ground stress, MPa; σ_{H} is the maximum horizontal ground stress, MPa;

The stress increments of casing, cement sheath, and formation at the boundary are equal and the displacement is continuous:

$$\begin{matrix}\{ \begin{matrix}Δ {σ}_{r 1} {\vert}_{r = {r}_{2}} = Δ {σ}_{r 2} {\vert}_{r = {r}_{2}} , Δ {τ}_{r θ 1} {\vert}_{r = {r}_{2}} = Δ {τ}_{r θ 2} {\vert}_{r = {r}_{2}} \\ Δ {σ}_{r 2} {\vert}_{r = {r}_{3}} = Δ {σ}_{r 3} {\vert}_{r = {r}_{3}} , Δ {τ}_{r θ 2} {\vert}_{r = {r}_{3}} = Δ {τ}_{r θ 3} {\vert}_{r = {r}_{3}}\end{matrix} & ( 8 ) \\ \{ \begin{matrix}Δ {u}_{r 1} {\vert}_{r = {r}_{2}} = Δ {u}_{r 2} {\vert}_{r = {r}_{2}} , Δ {u}_{θ1} {\vert}_{r = {r}_{2}} = Δ {u}_{θ 2} {\vert}_{r = {r}_{2}} \\ Δ {u}_{r 2} {\vert}_{r = {r}_{3}} = Δ {u}_{r 3} {\vert}_{r = {r}_{3}} , Δ {u}_{θ 2} {\vert}_{r = {r}_{3}} = Δ {u}_{θ 3} {\vert}_{r = {r}_{3}}\end{matrix} & ( 9 )\end{matrix}$$

S14. Bring it into the stress increment distribution function expression and displacement increment function expression of the casing-cement sheath-formation surrounding rock combination to obtain the casing-cement sheath-formation surrounding rock combination. Stress increment distribution and displacement increment distribution;

Cement sheath circumferential stress increment expression:

Δσ_{θ2}=ƒ(ΔP,Eᵢ,vᵢ,rⁱ,σᵥ,σ_{H},θ) (10)

The expression of radial stress increment of cement sheath:

Δσᵣ₂=g(Δp,Eᵢ,vᵢ,rⁱ,σᵥ,σ_{H},θ) (11)

S2. Establish a cement sheath integrity prediction model based on the cement sheath failure criterion and the stress increment distribution state of the cement sheath;

The cement sheath failure criteria include:

(1) Tensile Failure Criterion of Cement Sheath:

If it is required that the cement sheath does not undergo tensile failure, the following relationship should be satisfied:

Δσ_{θ2}<σₛ (12)

wherein: σₛ is the tensile strength of cement stone, MPa;

(2) Criteria for Peeling Failure of Cement Sheath Interface:

If it is required that no peeling failure occurs at the interface of the casing cement sheath, the following relationship should be satisfied:

Δσᵣ₂<σₜ (13);

wherein: σₜ is the yield strength of cement stone under confining pressure, MPa.

The cement sheath integrity prediction model includes a cement sheath tensile failure prediction model and a cement sheath interface peeling failure prediction model;

The tensile failure prediction model of the cement sheath is based on the tensile failure criterion of the cement sheath. If the circumferential stress increment of the inner wall of the cement sheath is less than the tensile strength of the cement stone, the tensile failure will not occur;

The cement-to-interface peeling failure prediction model is based on the cement-ring interface peeling failure criterion. If the radial displacement increment of the inner wall of the cement stone is less than the yield strength of the cement stone, the interface peeling failure of the cement stone will not occur.

S3. Establish a cement sheath integrity control method based on the cement sheath stress analysis model and the cement sheath integrity prediction model.

The cement sheath integrity control method includes:

Obtain current well casing, stratum surrounding rock mechanical parameters, size parameters, and wellhead pressure increment;

Set the Young's modulus and Poisson's ratio of the cement sheath mechanical parameters, simulate the stress state of the cement sheath according to the stress distribution model of the cement sheath, and calculate the circumferential stress increment and radial stress increment of the cement sheath.

Based on the cement sheath integrity prediction model described in step S2, the tensile strength of the cement stone should be greater than the circumferential stress on the inner wall of the cement sheath under the setting values of the cement stone Young's modulus and Poisson's ratio to avoid damage to the cement sheath. Increment, the yield strength of cement stone should be greater than the increase in radial stress on the inner wall of the cement sheath.

S4. Establish a functional relationship between cement stone mechanical parameters and strength parameters based on the cement sheath integrity control method.

The functional relationship between the mechanical parameters and strength parameters of the cement stone includes the binary function relationship between the cement stone Young's modulus, Poisson's ratio, and cement stone tensile strength, and the cement stone Young's modulus, Poisson's ratio, cement Binary function relationship between stone yield strength.

S5. Establish a cement stone performance index control chart based on the functional relationship between the cement stone mechanical parameters and strength parameters. The cement stone performance index control chart includes a chart to avoid tensile failure of the cement stone and a chart to avoid peeling failure of the cement stone.

### Embodiment

Taking the target well as an example, oil layer casing with an outer diameter of 127 mm and a wall thickness of 11.1 mm is generally used, and its internal pressure resistance is between 102.5 and 110.3 MPa. Since the construction pressure is generally not higher than 80% of the casing's internal pressure resistance, the wellhead pressure limit for hydraulic fracturing design is 80 MPa. Taking into account the requirements of pressure test, the design of cement stone performance that meets the wellhead pressure of 90 MPa is designed to avoid the failure of inter-segment isolation caused by tensile failure of the cement sheath and interface peeling failure. Other design parameters are shown in Table 1.

TABLE 1
Design parameters

| Parameters | Value | Unit |
|---|---|---|
| Open hole diameter | 165.1 | mm |
| Open hole diameter | 127 | mm |
| Outer diameter of casing | 11.1 | mm |
| Vertical stress | 83.98 | MPa |
| Maximum horizontal principal stress | 72.76 | MPa |
| Casing Poisson's ratio | 0.3 | — |
| Stratigraphic Poisson's Ratio | 0.218 | — |
| Young's modulus of casing | 206 | GPa |
| Young's modulus of formation | 21.55 | GPa |

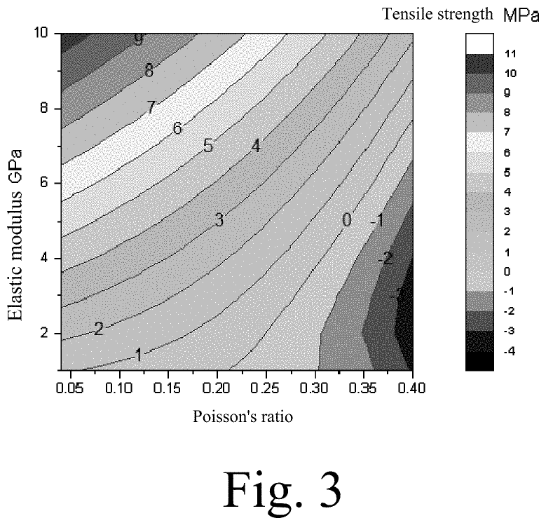

According to the simulation calculation results, when the wellhead construction pressure is 90 MPa, the cement stone properties is required to avoid tensile failure of the cement sheath are shown in FIG. 3.

It can be seen from FIG. 3 that when the Young's modulus of the cement stone is constant, the lower the Poisson's ratio, the higher the uniaxial tensile strength of the cement stone is required to avoid tensile failure of the cement sheath. When the Poisson's ratio of cement stone is constant, the greater the Young's modulus, the higher the uniaxial tensile strength needed to avoid tensile failure of the cement, and the Young's modulus has a much greater influence than Poisson's ratio. In order to avoid tensile failure of the cement sheath during large-scale fracturing, the performance of cementing cement stone needs to meet: when the Young's modulus of cement stone is 3.5 GPa and Poisson's ratio is 0.2, the corresponding tensile strength of cement stone Should not be less than 2.47 MPa. When the Young's modulus of cement stone is 4.5 GPa and Poisson's ratio is 0.2, the corresponding tensile strength of cement stone should not be less than 3.37 MPa. The elastic modulus of conventional cement stone is generally 10 GPa, Poisson's ratio is 0.3-0.4, and the corresponding tensile strength should not be less than 5.23 MPa.

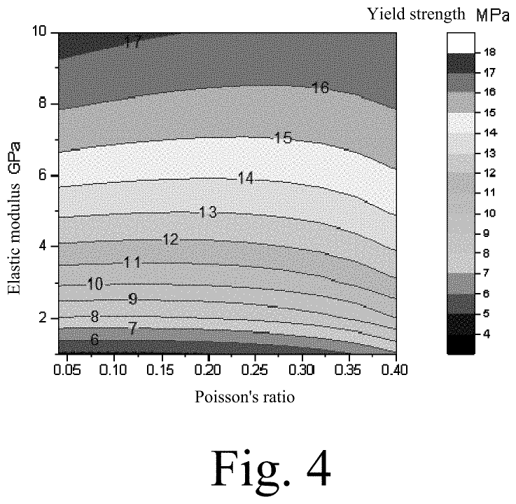

According to the simulation calculation results, when the wellhead construction pressure is 90 MPa, the cement stone properties required to avoid interfacial peeling failure of the cement sheath are shown in FIG. 4.

It can be seen from FIG. 4 that when the Young's modulus of the cement stone is constant, as the Poisson's ratio increases, the yield strength of the cement stone that requires no interfacial peeling failure of the cement stone increases first and then decreases. When the Poisson's ratio of cement stone is constant, the greater the Young's modulus is, the higher is the yield strength of cement stone that require no interface separation failure. In order to avoid the interface peeling failure of the cement sheath during the large-scale fracturing process, the performance of cementing cement stone needs to meet: when the Young's modulus of the cement stone is 3.5 GPa and the Poisson's ratio is 0.2, the corresponding cement stone yield The strength should not be less than 10.67 MPa. When the Young's modulus of the cement stone is 4.5 GPa and the Poisson's ratio is 0.2, the yield strength of the corresponding cement stone should not be less than 12.19 MPa.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method for establishing a chart for designing mechanical properties of cement stones in a large-scale fracturing oil well, comprising steps of:
S1: establishing a stress distribution model of a cement sheath based on a theory of elasticity and thick-walled cylinder;
S2: establishing a cement sheath integrity prediction model based on a cement sheath failure criterion and a stress increment distribution state of the cement sheath;
S3: establishing a cement sheath integrity control method based on a cement sheath stress analysis model and the cement sheath integrity prediction model;
S4: establishing a functional relationship between cement stone mechanical parameters and strength parameters based on the cement sheath integrity control method; and
S5: establishing a cement stone performance index control chart based on the functional relationship between the cement stone mechanical parameters and strength parameters.

2. The method for establishing the chart for designing mechanical properties of the cement stones in the large-scale fracturing oil well, as recited in claim 1, wherein the a specific establishing process of the step S1 comprises steps of:
S11: by Ariy's stress function, deriving the stress increment distribution function expression of the casing-cement sheath-stratum surrounding rock combination during the large-scale fracturing process based on the elasticity thick-walled cylinder theory;
S12: calculating the displacement increment function expression of the casing-cement sheath-stratum surrounding rock combination during the large-scale fracturing process according to the elasticity thick-walled cylinder theory; and
S13: calculating the unknown parameters in the stress increment distribution function expression and the displacement increment function expression using the conditions of equal stress increments at the boundary of the casing, cement sheath, and formation and continuous displacement, and then bringing them into the stress increment distribution function expression and displacement increment function expression of the casing-cement sheath-formation surrounding rock combination, and the stress increment distribution and displacement increase of the casing-cement sheath-formation surrounding rock combination is obtained.

3. The method for establishing the chart for designing mechanical properties of the cement stones in the large-scale fracturing oil well, as recited in claim 2, wherein the stress increment distribution function expression comprises a circumferential stress increment expression, a radial stress increment expression, and a shear stress increment expression.

4. The method for establishing the chart for designing mechanical properties of the cement stones in the large-scale fracturing oil well, as recited in claim 1, wherein the cement sheath failure criterion comprises a cement sheath tensile failure criterion and a cement sheath interface peeling failure criterion.

5. The method for establishing the chart for designing mechanical properties of the cement stones in the large-scale fracturing oil well, as recited in claim 4, wherein the cement sheath integrity prediction model comprises a cement sheath tensile failure prediction model and a cement sheath interface peeling failure prediction model;
the tensile failure prediction model of the cement sheath is based on the tensile failure criterion of the cement sheath; if the circumferential stress increment of the inner wall of the cement sheath is smaller than the tensile strength of the cement stone, the tensile failure will not occur;
the cement-to-interface peeling failure prediction model is based on the cement sheath interface peeling failure criterion, if the radial displacement increment of the inner wall of the cement stone is less than the yield strength of the cement stone, the interface peeling failure of the cement stone will not occur.

6. The method for establishing the chart for designing mechanical properties of the cement stones in the large-scale fracturing oil well, as recited in claim 1, wherein the cement sheath integrity control method comprises following steps of:
A: obtaining current well casing, stratum surrounding rock mechanical parameters, size parameters, and wellhead pressure increment;
B: setting the Young's modulus and Poisson's ratio of the mechanical parameters of the cement sheath, simulate the stress state of the cement sheath according to the stress distribution model of the cement sheath, and calculate the circumferential stress increment and radial stress increment of the cement sheath; and
C: based on the cement sheath integrity prediction model, under the current value of cement stone Young's modulus and Poisson's ratio, in order to avoid damage to the cement sheath, setting the cement stone tensile strength greater than the circumferential stress increment of the inner wall of the cement sheath, and the cement stone yields, and the strength greater than the increase in radial stress on the inner wall of the cement sheath.

7. The method for establishing the chart for designing mechanical properties of the cement stones in large-scale fracturing oil well, as recited in claim 1, wherein the functional relationship between the mechanical parameters and strength parameters of the cement stone includes the binary function relationship between the cement stone Young's modulus, Poisson's ratio, and the cement stone's tensile strength, and the cement stone Young's modulus, Poisson's ratio, Binary function relationship between the yield strength of cement stone.

8. The method for establishing the chart for designing mechanical properties of the cement stones in the large-scale fracturing oil well, as recited in claim 5, wherein the cement stone performance index control plate comprises a plate to avoid tensile failure of cement stone and a plate to avoid peeling failure of cement stone.
